(19) United States
(12) Patent Application Publication
(10) Pub. No.: US 2017/0304406 A1
(43) Pub. Date: Oct. 26, 2017

(54) NEW INDICATION FOR ALPHA-MSH ANALOGUES

(71) Applicant: CLINUVEL AG, Zug (CH)

(72) Inventor: Philippe WOLGEN, Zug (CH)

(21) Appl. No.: 15/522,260

(22) PCT Filed: Oct. 28, 2015
(86) PCT No.: PCT/EP2015/075017

§ 371 Date: Apr. 26, 2017

(30) Foreign Application Priority Data
Oct. 28, 2014 (EP) 14190766.7

(51) Int. Cl. A61K 38/34; A61K 9/00

(52) U.S. Cl. CPC A61K 38/34; A61K 9/0019

(57) ABSTRACT

The present invention is directed to alpha-MSH analogues for treatment of neurodegenerative disorders.

### TECHNICAL FIELD

The present invention is directed to a compound for use in treatment of a human subject with a medical indication, to a method of treating neurodegenerative disorders, and to a use of an alpha-MSH analogue for the manufacture of a medicament for the treatment of a human subject.

### BACKGROUND TO THE INVENTION

Many studies have been conducted to investigate cause and treatment of Central Nervous System (CNS) disorders. Most studies are based on animal models. However, there remains a need for further improvements, including efficacy and safety aspects.

### SUMMARY OF THE INVENTION

According to the invention, we have found surprising benefits of the particular use of alpha-MSH analogues in treatment and/or prevention of Central Nervous System (CNS) disorders. Accordingly, the present invention relates to an alpha-MSH analogue for use in treatment of a human subject with a neurodegenerative disorder wherein the interval between subsequent administrations of the alpha-MSH analogue is between at least 6 weeks and at most 8 weeks. Preferably, the disorder is a juvenile form of the neurodegenerative disorder.

In one aspect, the neurodegenerative disorder is Multiple Sclerosis. In another aspect, the neurodegenerative disorder is dementia. In another aspect, the neurodegenerative disorder is Alzheimer's Disease. In another aspect, the neurodegenerative disorder is Parkinson's Disease. In another aspect, the neurodegenerative disorder is Amyotrophic Lateral Sclerosis (ALS). In another aspect, the neurodegenerative disorder is Huntington's Disease.

Preferably, the alpha-MSH analogue is administered systemically. Preferably, the alpha-MSH analogue is administered subcutaneously. Preferably, the alpha-MSH analogue is present in the blood plasma of the subject at a level of between at least 0.01 ng/ml to at most 10 ng/ml for a period of at least 2 days after administration. Preferably, the alpha-MSH analogue is a derivative of alpha-MSH which exhibits agonist activity for the melanocortin-1-receptor (MC1R), the receptor to which alpha-MSH binds to initiate the production of melanin within a melanocyte. Preferably, the alpha-MSH analogue is afamelanotide.

In another aspect, the invention relates to a method of treating neurodegenerative disorders by administering an alpha-MSH analogue to a human subject suffering from neurodegenerative disorder, wherein the interval between subsequent administrations of the alpha-MSH analogue is at least 6 weeks and at most 8 weeks.

In another aspect, the invention relates to the use of an alpha-MSH analogue for the manufacture of a medicament for the treatment of a human subject with a neurodegenerative disorder wherein the interval between subsequent administrations of the alpha-MSH analogue is at least 6 weeks and at most 8 weeks.

We have surprisingly found that the invention allows for effective yet safe and convenient treatment of neurodegenerative disorders using alpha-MSH analogues.

### DETAILS DESCRIPTION OF THE INVENTION

For the purpose of this invention, treatment is defined as encompassing prevention of a disorder.

According to the invention, we have surprisingly found that alpha-MSH analogues are effective in treatment of CNS disorder of human subjects, preferably neurodegenerative disorders. For the purpose of this invention, neurodegenerative disorders are characterized by progressive nervous system dysfunction. These disorders are often associated with atrophy of the affected central or peripheral structures of the nervous system. According to the invention, neurodegenerative disorders preferably include the diseases of Multiple Sclerosis, Alzheimer's Disease and other dementias, Parkinson's Disease, Amyotrophic Lateral Sclerosis (ALS) and which is also called Lou Gehrig's Disease, Huntington's Disease, Degenerative Nerve Diseases, Encephalitis, Epilepsy, Genetic Brain Disorders, Head and Brain Malformations, Hydrocephalus, Stroke, and Prion Diseases.

Accordingly, in one aspect, the present invention is directed to the above-mentioned neurodegenerative disorders, preferably the juvenile form thereof, each separately and as a group.

In another aspect, the present invention is directed to Multiple Sclerosis, preferably the juvenile form which is defined according to the invention as occurring before the age of 18 years. In another aspect, the present invention is directed to dementia, preferably the juvenile form. In another aspect, the present invention is directed to Alzheimer's Disease, preferably the juvenile form which is defined according to the invention as occurring before the age of 65 years. In another aspect, the present invention is directed to Parkinson's Disease, preferably the juvenile form which is defined according to the invention as occurring before the age of 20 years. In another aspect, the present invention is directed to Amyotrophic Lateral Sclerosis (ALS) which is often called Lou Gehrig's Disease, and preferably the juvenile form which is defined according to the invention as occurring before the age of 25 years. In another aspect, the present invention is directed to Huntington's Disease, preferably the juvenile form which is defined according to the invention as occurring before the age of 20 years.

According to the invention, the human subject is preferably exposed to alpha-MSH analogue at a blood plasma level of at least 0.01 ng/ml, more preferably at least 0.1 ng/ml, most preferably at least 1 ng/ml and preferably at most 20 ng/ml, more preferably at most 15 ng/ml, most preferably at most 10 ng/ml. Preferably, exposure is for at least 1 day, more preferably at least 2 days, more preferably at least 5 days and preferably at most 30 days, more preferably at most 20 days, most preferably at most 15 days and particularly preferred for at most 10 days, for instance for 7 days or for 10 days. It will be understood that these alpha-MSH analogue blood plasma levels are achieved after each alpha-MSH analogue administration. As will be understood by a skilled person in the art, after initial alpha-MSH analogue release and absorption by the subject into the blood plasma, the alpha-MSH analogue will be present in the blood plasma of the subject at a level and the time period indicated. Thus, the alpha-MSH analogue is administered in an amount that results in the blood plasma levels indicated. Accordingly, the human subject is subjected to the blood plasma levels indicated.

It is preferred according to the present invention to administer the alpha-MSH analogue systemically. Preferably, the alpha-MSH analogue is administered subcutaneously. Preferred systemic administration of the alpha-MSH analogue of the invention is by way of an injection, more preferably by way of a subcutaneously injected implant. Preferred systemic administration is by way of a controlled-release formulation.

According to a preferred treatment of the invention, the alpha-MSH analogue is at least 2 times administered subsequently to a subject, more preferably at least 3 times, most preferably at least 5 times and for instance up to 20 times. Preferably, the interval between subsequent administrations is at least 2 weeks, more preferably at least 4 weeks, most preferably at least 5 weeks, and most preferably at least 6 weeks and preferably at most 10 weeks, more preferably at most 9 weeks, most preferably at most 8 weeks. According to the invention, a particularly preferred range for the interval between subsequent administrations of the alpha-MSH analogue is from 6 to 8 weeks. It will be understood that for the purpose of the invention, intervals are separate and subsequent and do not overlap.

According to one aspect, the invention is directed to alpha-MSH analogues. The term “alpha-MSH analogue” as used herein is defined as a derivative of alpha-MSH which exhibits agonist activity for the melanocortin-1-receptor (MC1R), the receptor to which alpha-MSH binds to initiate the production of melanin within a melanocyte. Such alpha-MSH analogues include derivatives in which (i) one or more amino acid residues are deleted from the native alpha-MSH molecule at the N-terminal end, the C-terminal end, or both; and/or (ii) one or more amino acid residues of the native alpha-MSH molecule are replaced by another natural, non-natural or synthetic amino acid residue; and/or (iii) an intra-molecular interaction forms as a cyclic derivative. Several derivatives of alpha-MSH have been synthesized. In one aspect of the present invention, the alpha-MSH analogues described in U.S. Pat. Nos. 4,457,864, 4,485,039, 4,866,038, 4,918,055, 5,049,547, 5,674,839 and 5,714,576 and Australian Patents Nos. 597630 and 618733, which are herein incorporated by reference for their teachings with respect to alpha-MSH analogues and their synthesis thereof, can be used herein. The alpha-MSH analogue may be used as such or in the form of a pharmaceutically acceptable salt thereof. Preferred examples of such salts are acetate, trifluoroacetate, sulphate, and chloride salts. The acetate salt is generally most preferred.

Preferably, according to the invention, the alpha-MSH analogue is a non-radiation emitting analogue, i.e. the compound is not radioactive that can be damaging to the body. In other words, the alpha-MSH analogue emits low and preferably no radiation including alpha, beta and/or gamma radiation at the level or lower than average background radiation levels.

In one aspect of the invention, the alpha-MSH analogue is selected from the group consisting of:

(a) compounds of the formula:

Ac-Ser-Tyr-Ser-M-Gln-His-D-Phe-Arg-Trp-Gly-Lys-
Pro-Val-NH₂

wherein M is Met, Nle or Lys; and

(b) compounds of the formula:

R₁-W-X-Y-Z-R₂

wherein

R₁ is absent, n-pentadecanoyl, Ac, 4-phenylbutyryl, Ac-Gly-, Ac-Met-Glu, Ac-Nle-Glu-, or Ac-Tyr-Glu-;

W is -His- or-D-His-;

X is -Phe-, -D-Phe-, -Tyr-, -D-Tyr-, or -(pN0₂)D-Phe⁷-;

Y is -Arg- or -D-Arg-;

Z is -Trp- or -D-Trp-; and

R₂ is -NH₂; -Gly-NH₂; or-Gly-Lys-NH₂, as disclosed in Australian Patent No. 597630.

In another aspect, the alpha-MSH analogue may be a linear analogue as disclosed in U.S. Pat. No. 5,674,839, and selected from the group consisting of:

Ac-Ser-Tyr-Ser-Nle-Glu-His-D-Phe-Arg-Trp-Lys-Gly-
Pro-Val-NH₂,
Ac-Ser-Tyr-Ser-Nle-Asp-His-D-Phe-Arg-Trp-Lys-Gly-
Pro-Val-NH₂,
Ac-Nle-Glu-His-D-Phe-Arg-Trp-Lys-Gly-Pro-Val-NH₂,
Ac-Nle-Asp-His-D-Phe-Arg-Trp-Lys-Gly-Pro-Val-NH₂,
Ac-Nle-Asp-His-D-Phe-Arg-Trp-Gly-NH₂,
Ac-Nle-Glu-His-D-Phe-Arg-Trp-Lys-NH₂,
Ac-Nle-Asp-His-D-Phe-Arg-Trp-Lys-NH₂,
Ac-Nle-Glu-His-D-Phe-Arg-Trp-Orn-NH₂,
Ac-Nle-Asp-His-D-Phe-Arg-Trp-Orn-NH₂,
Ac-Nle-Glu-His-D-Phe-Arg-Trp-Dab-NH₂,
Ac-Nle-Asp-His-D-Phe-Arg-Trp-Dab-NH₂,
Ac-Nle-Glu-His-D-Phe-Arg-Trp-Dpr-NH₂,
Ac-Nle-Glu-His-Phe-Arg-Trp-Lys-NH₂,
and
Ac-Nle-Asp-His-Phe-Arg-Trp-Lys-NH₂.

In another aspect, the alpha-MSH analogue may also be a cyclic analogue as disclosed in U.S. Pat. No. 5,674,839, selected from the group consisting of:

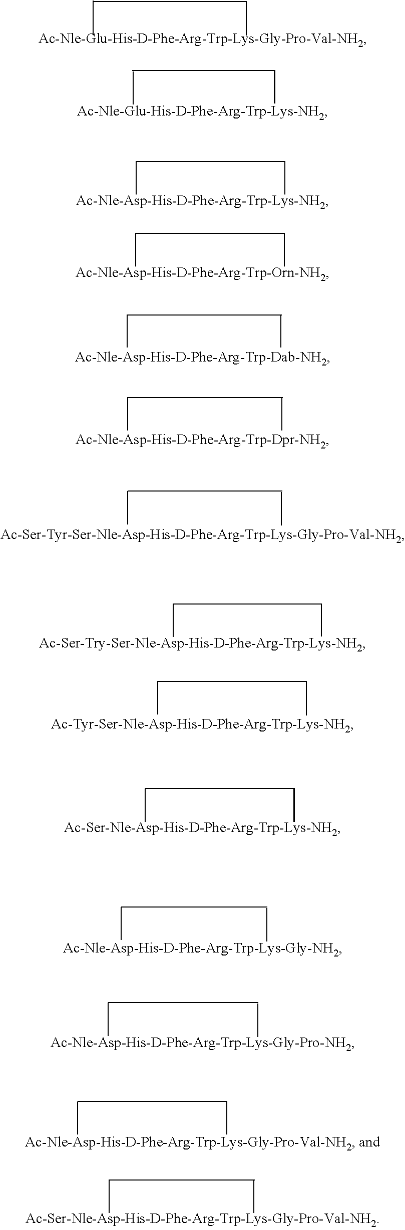

wherein Ala=alanine, Arg=arginine, Dab-2,4-diaminobutyric acid, Dpr=2,3-diaminopropionic acid, Glu=glutamic acid, Gly=glycine, His=histidine, Lys=lysine, Met=methionine, Nle=norleucine, Orn=ornithine, Phe=phenylalanine, (pNO2)Phe=paranitrophenylalanine, Plg=phenylglycine, Pro=proline, Ser=serine, Trp=tryptophan, TrpFor=N¹ formyl-tryptophan, Tyr=tyrosine, Val=valine.

All peptides are written with the acyl-terminal end at the left and the amino terminal end to the right; the prefix “D” before an amino acid designates the D-isomer configuration, and unless specifically designated otherwise, all amino acids are in the L-isomer configuration.

In another aspect, the alpha-MSH analogue is preferably selected from the group consisting of:

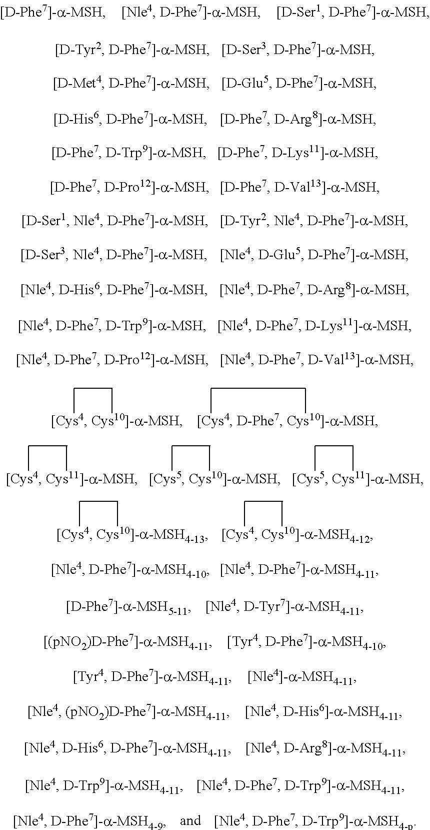

Preferred alpha-MSH analogues thereof are selected from the group consisting of:

[Nle⁴, D-Phe⁷]-α-MSH₄₋₁₀,

[Nle⁴, D-Phe⁷]-α-MSH₄₋₁₁,

[Nle⁴, D-Phe⁷, D-Trp⁹]-α-MSH₄₋₁₁, and

[Nle⁴, D-Phe⁷]-α-MSH₄₋₉.

In another aspect, the alpha-MSH analogue is a cyclic peptide of formula (I):

Z-Xaa¹-Xaa²-Xaa³-Xaa⁴-Xaa⁵-Xaa⁶-Xaa⁷-Y (I)

or a pharmaceutically acceptable salt thereof, wherein:

Z is H or an N-terminal group wherein the N-terminal group is preferably a C₁ to C₁₇ acyl group, wherein the C₁ to C₁₇ comprises a linear or branched alkyl, cycloalkyl, alkylcycloalkyl, aryl or alkylaryl, a linear or branched C₁ to C17 alkyl, aryl, heteroaryl, alkene, alkenyl, or aralkyl chain or an N-acylated linear or branched C₁ to C₁₇ alkyl, aryl, heteroaryl, alkene, alkenyl, or aralkyl chain and more preferably is a C₁ to C₇ acyl group;

Xaa¹ is optionally present, and if present is from one to three L- or D-isomer amino acid residues, and preferably an amino with a side chain including a linear or branched alkyl, cycloalkyl, cycloheteroalkyl, aryl or heteroaryl, and more preferably is an L- or D-isomer of Nle;

Xaa² and Xaa⁶ are L- or D-isomer amino acids wherein the side chains thereof comprise a cyclic bridge, and, preferably, one of Xaa² and Xaa⁶ is an L- or D-isomer of Asp, hGlu or Glu and the other of Xaa² and Xaa⁶ is an L- or D-isomer of Lys, Orn, Dab or Dap or, in an alternative preferred aspect, Xaa² and Xaa⁶ are each Cys, D-Cys, Pen or D-Pen;

Xaa³ is L- or D-Pro, optionally substituted with hydroxyl, halogen, sulfonamide, alkyl, —O-alkyl, aryl, alkyl-aryl, alkyl-O-aryl, alkyl-O-alkyl-aryl, or —O-aryl, or Xaa³ is an L- or D-isomer of an amino acid with a side chain including at least one primary amine, secondary amine, alkyl, cycloalkyl, cycloheteroalkyl, aryl, heteroaryl, ether, sulfide, or carboxyl and preferably is an L-or D-isomer of His;

Xaa⁴ is an L- or D-isomer amino acid with a side chain including phenyl, naphthyl or pyridyl, optionally wherein the ring is substituted with one or more substituents independently selected from halo, (C₁-C₁₀)alkyl-halo, (C₁-C₁₀)alkyl, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, and alkoxy-carbonyl, and is preferably D-Phe, optionally substituted with one or more substituents independently selected from halo, (C₁-C₁₀)alkyl-halo, (C₁-C₁₀)alkyl, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, and alkoxy-carbonyl; Xaa⁵ is L- or D-Pro or an L- or D-isomer amino acid with a side chain including at least one primary amine, secondary amine, guanidine, urea, alkyl, cycloalkyl, cycloheteroalkyl, aryl, heteroaryl, or ether and preferably is an L- or D-isomer of Arg, Lys, Orn, Dab or Dap;

Xaa⁷ is optionally present, and if present is from one to three L- or D-isomer amino acid residues, and is preferably an amino acid with a side chain including at least one aryl or heteroaryl, optionally substituted with one or more ring substituents, and when one or more substituents are present, are the same or different and independently hydroxyl, halogen, sulfonamide, alkyl, —O-alkyl, aryl, or —O-aryl, and more preferably is an L- or D-isomer of Trp, Nal 1 or Nal 2; and

Y is a C-terminal group and in another aspect preferably a hydroxyl, an amide, or an amide substituted with one or two linear or branched C₁ to C₁₇ alkyl, cycloalkyl, aryl, alkyl cycloalkyl, aralkyl, heteroaryl, alkene, alkenyl, or aralkyl chains.

Preferred cyclic alpha-MSH analogues are Ac-Nle-cyclo(Glu-His-D-Phe-Arg-Dab)-Trp-N H₂ and Ac-Nle-cyclo(Glu-His-D-Phe-Arg-Dap)-Trp-N H₂.

According to this aspect and in addition to the above defined amino acids, the amino acids are defined in US2013/0296256 pages 5 and 6 which are incorporated herein by reference. Further, the terms “a,a-disubstituted amino acid”, “N-substituted amino acid”, “alkane”, “alkene”, “alkenyl”, “alkyl”, “alkyne”, “aryl”, “aralkyl”, “aliphatic”, “acyl”, “acylated”, “omega amino aliphatic chain”, “heteroaryl”, “amide”, “imide”, “amine”, “nitrile”, and “halogen” are defined on pages 6 and 7 thereof and are also incorporated herein by reference.

According to the present invention, the most preferred alpha-MSH analogue is [Nle⁴, D-Phe⁷]-alpha-MSH. This preferred compound is sometimes referred to as NDP-MSH. It is also generically known as afamelanotide, which is available as an implant formulation under the trademark SCENESSE®.

Preferably, the alpha-MSH analogue is administered in a composition. Preferably, the composition is a slow release formulation, resulting in longer and/or more controlled exposure of the body to the drug. Most preferably, the composition is an implant. In one preferred embodiment, the alpha-MSH analogue is administered in a prolonged release formulation such as described in US2008305152 (equivalent to WO2006/012667), the disclosure of which is included herein by reference.

The composition preferably comprises at least 5 mg of the alpha-MSH analogue, more preferably at least 10 mg and preferably at most 30 mg, more preferably at most 25 mg of the alpha-MSH analogue. Particularly preferred amounts are 20 mg or 16 mg of the alpha-MSH analogue of which 16 mg of the alpha-MSH analogue is the most preferred.

Preferably, the composition comprises a controlled release formulation. In one aspect according to the present invention, the implant (or rod) comprises a biodegradable polymer, wherein the alpha-MSH analogue is imbedded within the implant. In one aspect, the alpha-MSH analogue is encapsulated in an implant composed of poly-(lactide-co-glycolide), poly-(lactide), poly-(glycolide) or a mixture thereof. Lactide/glycolide polymers for drug-delivery formulations are typically made by melt polymerization through the ring opening of lactide and glycolide monomers. Some polymers are available with or without carboxylic acid end groups. When the end group of the poly-(lactide-co-glycolide), poly-(lactide), or poly-(glycolide) is not a carboxylic acid, for example, an ester, then the resultant polymer is referred to herein as blocked or capped. The unblocked polymer, conversely, has a terminal carboxylic group. In one aspect, linear lactide/glycolide polymers are used; however star polymers can be used as well. In certain aspects, high molecular weight polymers can be used for medical devices, for example, to meet strength requirements. The lactide portion of the polymer has an asymmetric carbon. Commercially racemic DL-, L-, and D-polymers are available. The L-polymers are more crystalline and resorb slower than DL-polymers. In addition to copolymers comprising glycolide and DL-lactide or L-lactide, copolymers of L-lactide and DL-lactide are available. Additionally, homo-polymers of lactide or glycolide are available. In the case when the biodegradable polymer is poly-(lactide-co-glycolide), poly-(lactide), or poly-(glycolide), the amount of lactide and glycolide in the polymer can vary. In one aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In one aspect, the biodegradable polymer can be poly-(lactide), 85:15 poly-(lactide-co-glycolide), 75:25 poly-(lactide-co-glycolide), or 65:35 poly-lactide-co-glycolide) where the ratios are mole ratios.

In one aspect, when the biodegradable polymer is poly-(lactide-co-glycolide), poly-(lactide), or poly-(glycolide), the polymer has an intrinsic viscosity of from 0.15 to 1.5 dL/g, 0.25 to 1.5 dL/g, 0.25 to 1.0 dL/g, 0.25 to 0.8 dL/g, 0.25 to 0.6 dL/g, or 0.25 to 0.4 dL/g as measured in chloroform at a concentration of 0.5 g/dL at 30° C.

The implant preferably comprises alpha-MSH analogue in an amount of from 5% to 60%, more preferably from 10% to 50%, most preferably from 15% to 40%, and in particularly preferred from 15% to 30% by weight of the implant. Preferred implants are described in US2008/0305152 incorporated herein by reference. A preferred implant comprising afamelanotide is available under the name of SCENESSE® in Italian and Swiss markets.

Other pharmaceutically-acceptable components can be encapsulated or incorporated in the composition or in the implant in combination with the alpha-MSH analogue. For example, the pharmaceutically-acceptable component can include a fatty acid, a sugar, a salt, a water-soluble polymer such as polyethylene glycol, a protein, polysacharride, or carboxmethyl cellulose, a surfactant, a plasticizer, a high- or low-molecular-weight porosigen such as polymer or a salt or sugar, or a hydrophobic low-molecular-weight compound such as cholesterol or a wax.

1. Alpha-MSH analogue for use in treatment of a human subject with a neurodegenerative disorder wherein the interval between subsequent administrations of the alpha-MSH analogue is between at least 6 weeks and at most 8 weeks.

2. Compound for use according to claim 1, wherein the disorder is a juvenile form of the neurodegenerative disorder.

3. Compound for use according to claim 1, wherein the neurodegenerative disorder is Multiple Sclerosis.

4. Compound for use according to claim 1, wherein the neurodegenerative disorder is dementia.

5. Compound for use according to claim 1, wherein the neurodegenerative disorder is Alzheimer's Disease.

6. Compound for use according to claim 1, wherein the neurodegenerative disorder is Parkinson's Disease.

7. Compound for use according to claim 1, wherein the neurodegenerative disorder is Amyotrophic Lateral Sclerosis (ALS).

8. Compound for use according to claim 1, wherein the neurodegenerative disorder is Huntington's Disease.

9. Compound for use according to claim 1, wherein the alpha-MSH analogue is administered systemically.

10. Compound for use according to claim 1, wherein the alpha-MSH analogue is administered subcutaneously.

11. Compound for use according to claim 1, the alpha-MSH analogue is present in the blood plasma of the subject at a level of between at least 0.01 ng/ml to at most 10 ng/ml for a period of at least 2 days after administration.

12. Compound for use according to claim 1, wherein the alpha-MSH analogue is a derivative of alpha-MSH which exhibits agonist activity for the melanocortin-1-receptor (MC1R), the receptor to which alpha-MSH binds to initiate the production of melanin within a melanocyte.

13. Compound for use according to claim 1, wherein the alpha-MSH analogue is afamelanotide.

14. Method of treating neurodegenerative disorders by administering an alpha-MSH analogue to a human subject suffering from neurodegenerative disorder, wherein the interval between subsequent administrations of the alpha-MSH analogue is at least 6 weeks and at most 8 weeks.

15. Use of an alpha-MSH analogue for the manufacture of a medicament for the treatment of a human subject with a neurodegenerative disorder wherein the interval between subsequent administrations of the alpha-MSH analogue is at least 6 weeks and at most 8 weeks.
